(19) United States
(12) Patent Application Publication
(10) Pub. No.: US 2025/0177172 A1
(43) Pub. Date: Jun. 5, 2025

(54) PROSTHETIC SOCKET WITH INFLATABLE BLADDERS AND AUTOMATED BLADDER INFLATION AND PRESSURE CONTROL

(71) Applicant: D. Barry McCoy, Coventry, RI (US)

(72) Inventor: D. Barry McCoy, Coventry, RI (US)

(21) Appl. No.: 19/043,182

(22) Filed: Jan. 31, 2025

Related U.S. Application Data
(63) Continuation-in-part of application No. 18/498,095, filed on Oct. 31, 2023.
(60) Provisional application No. 63/548,928, filed on Feb. 2, 2024.
(60) Provisional application No. 63/381,985, filed on Nov. 2, 2022.

(51) Int. Cl. A61F 2/78; A61F 2/70; A61F 2/74; A61F 2/80; G16H 40/63

(52) U.S. Cl. CPC A61F 2/7843; A61F 2/741; A61F 2/80; G16H 40/63; A61F 2002/704; A61F 2002/705; A61F 2002/707; A61F 2002/785

(57) ABSTRACT

According to embodiments of the invention a novel prosthetic socket may include one or more inflatable pressure bladders disposed within the socket which may be controlled to be set at a desired pressured by the patient, and then automatically monitored and controlled to maintain the set pressure. The user may further incrementally increase or decrease the pressure as needed for different activities. A control system may comprise an external control device, such as a cell phone/tablet running a proprietary control and monitoring application, and may further include a back-end server which stores patient data, operating values and long term monitoring data.

### CROSS-REFERENCE TO RELATED APPLICATIONS

This application is a Continuation-In-Part application of pending U.S. patent application Ser. No. 18/498,095 filed Oct. 31, 2023, which claims the benefit of U.S. Provisional Patent Application No. 63/381,985 filed Nov. 2, 2022 the entire contents of which are incorporated herein by reference.

This application also claims the benefit of U.S. Provisional Patent Application No. 63/548,928, filed Feb. 2, 2024, the entire contents of which is incorporated herein by reference.

### BACKGROUND OF THE DISCLOSURE

### (1) Field of the Invention

The instant invention relates in one aspect to scoliosis bracing and treatment protocols, and more specifically to a novel scoliosis brace having inflatable pressure bladders which are cyclically controlled to increase and decrease pressure over a given treatment period and a treatment protocol which monitors and tracks the quality and quantity of brace “wear time” to modify and/or increase bladder pressures and cycle times over a long-term bracing period. The instant invention also generally relates to prostheses and more specifically to a novel prosthetic socket and suspension system having inflatable pressure bladders for which a desired pressure can set by the user and then automatically controlled to maintain a constant set pressure over a given wear period.

### (2) Description of Related Art

Idiopathic scoliosis (“IS”) is a curvature of the spine which may include vertebral rotation, affecting the rib cage and presenting deformities of the trunk. Treatment modalities for scoliosis are based on patient's physiologic maturity, curve severity, curve location, surface deformities, and the risk of progression.

Bracing is a common non-surgical method to control curve progression during the high-risk growth phase of adolescent patients. A brace typically comprises a hard plastic shell with internal localized pressure pads to provide passive mechanical support to the spine. A properly fitted brace (if used consistently) progressively applies corrective lateral pressure to targeted areas of the spinal column and in theory should reduce the curve with the goal of treatment being generally accepted to prevent further curve progression without surgical intervention.

To be effective, the brace must be worn consistently as prescribed and until the child has completed growth. However, many recent studies have questioned the effectiveness of bracing and therefore the efficacy of bracing still remains a controversial topic among pediatric orthopedic surgeons.

The most important determinant of the effectiveness of bracing management is compliance, both in terms of “quantity” (i.e. wearing the brace for the prescribed amount of time per day) and “quality” (i.e. while being worn the brace is providing the intended curve correction). To monitor compliance, the most common method is an honor system where the patient/family reports wear time. Other electronic monitoring methods, such as temperature sensors, and pressure switches and force sensors have been used to study compliance but these methods have significant practical limitations and none measured both the quantity and quality of brace usage at the same time. Even if wear time “quantity” is accurately reported and tracked over time, the “quality” of the wear time has been difficult to validate. Without known pressure values, meaningful conclusions about the effectiveness of bracing for any particular patient are difficult to draw.

Prosthesis fitting also presents many of the same fitting and wear issues as scoliosis bracing. Prostheses play an important role in allowing amputee patients to perform daily activities. Patients with amputated lower limbs rely on prostheses for virtually every type of activity and may need to wear their prosthesis for extended times over the course of a day throughout a variety of different situations, including standing, sitting, driving, walking, exercising, etc. Proper fit and conformance with the amputated limb is critical for comfort and effectiveness.

Prosthesis fitting is mainly determined by socket design and shape and by a suspension system within the socket. The residual limb is essentially a bone or bones that is/are suspended in a mass of flesh. Trying to effectively control the motion of the bone relative to the surrounding flesh and relative to the socket has always been a challenge.

Furthermore, it is well know that the volume of the residual limb can vary as much as +/−10% over the course of a day depending on activity, water intake, eating and other environmental factors. Obviously, these constant changes in volume alter surface contact area between the residual limb and the interior of the prosthetic socket and can cause either loose fit or pressure points leading to chafing, blisters and discomfort as well as imbalances in gait and muscle and/or muscle atrophy as well as the development of calluses and scar tissue.

Contouring of the physical socket has been done for years to try to effectively capture the individual anatomy. However, there are limits to what can be done with a hard socket interface.

Prior art solutions have involved sizing inserts, as well as manually adjustable tightening mechanisms. However, these manual systems require constant vigilance and adjustment. Furthermore, it is nearly impossible to compensate for varying volume changes in specific area of the residual limb.

Inflatable air bladders have also been suggested in the art. However, the existing systems have predominantly implemented manual inflation systems, and because of the constantly changing volume of the residual limb, always require monitoring and manual inflation and deflation.

Vacuum socket systems have also been suggested in the prior art where a vacuum pump is attached to an interior socket liner and used to create a vacuum between the residual limb and the socket to enable the socket to be more effectively coupled to the limb. Automatic vacuum control and feedback can be provided.

### SUMMARY OF THE DISCLOSURE

According to some embodiments of the invention, a novel scoliosis brace may include one or more inflatable pressure bladders in the thoracic and/or lumbar area(s) which are cyclically controlled and monitored to increase and decrease pressure over a given treatment period. A treatment protocol monitors and tracks the quality and quantity of brace “wear time” to provide better data for modifying and/or increasing bladder pressures and cycle times over a long-term bracing period.

Generally, the system comprises a novel scoliosis brace with an integrated inflatable bladder system, and external control device (cell phone/tablet) running a proprietary control and monitoring application, and may further include a back-end server which stores patient data, operating values and long term recorded wear data.

According to some embodiments of the invention, a novel prosthetic socket may include one or more inflatable pressure bladders disposed within the socket which may be controlled to be set at a desired pressured by the patient, and then automatically monitored and controlled to maintain the set pressure. The user may further incrementally increase or decrease the pressure as needed for different activities.

The prosthetic inflation and pressure control system may comprise an external control device, such as a cell phone/tablet running a proprietary control and monitoring application, and may further include a back-end server which stores patient data, operating values and long term monitoring data.

While embodiments of the invention have been described as having the features recited, it is understood that various combinations of such features are also encompassed by particular embodiments of the invention and that the scope of the invention is limited by the claims and not the description.

### BRIEF DESCRIPTION OF THE DRAWING FIGURES

While the specification concludes with claims particularly pointing out and distinctly claiming particular embodiments of the instant invention, various embodiments of the invention can be more readily understood and appreciated from the following descriptions of various embodiments of the invention when read in conjunction with the accompanying drawings in which:

FIG. 1 is a front view of an exemplary embodiment of a scoliosis bracing system in accordance with the teachings of the present disclosure;

FIG. 2 is a rear view thereof showing the bladder inflation pump and control;

FIG. 3 is a bottom view thereof shower the lumbar bladder positioned between the padding and the outer shell;

FIG. 4 is a schematic block diagram of the bladder inflation and control system;

FIG. 5 is a schematic block diagram of the associated wireless control device;

FIG. 6 is a schematic block diagram of the overall communication system including a remote server;

FIG. 7 is an exemplary table showing one example of progressive cycle times and pressures;

FIG. 8 is a perspective view of another exemplary embodiment with the air supply tubes positioned between the outer shell and the foam padding;

FIG. 9 is a front view of an exemplary embodiment of a “below the knee” prostheses in accordance with the teachings of the present disclosure;

FIG. 10 is a top view thereof showing placement of the bladder inside the socket;

FIG. 11 is a top view of an “above the knee” prostheses in accordance with the teachings of the present disclosure;

FIG. 12 is another top view thereof;

FIG. 13 illustrates an exemplary control device with adjustment settings;

FIG. 14 is a schematic block diagram of the bladder inflation and control system;

FIG. 15 is a schematic block diagram of the associated wireless control device; and

FIG. 16 is a schematic block diagram of the overall communication system including a remote server.

### DETAILED DESCRIPTION OF THE EXEMPLARY EMBODIMENTS

Certain exemplary embodiments will now be described to provide an overall understanding of the principles of the structure, function, manufacture, and use of the device and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present disclosure. Further, in the present disclosure, like-numbered components of the embodiments generally have similar features, and thus within a particular embodiment each feature of each like-numbered component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Further, to the extent that directional terms like top, bottom, up, or down are used, they are not intended to limit the systems, devices, and methods disclosed herein. A person skilled in the art will recognize that these terms are merely relative to the system and device being discussed and are not universal.

According to some embodiments of the invention, a novel scoliosis brace 10 includes inflatable pressure bladders which are cyclically controlled and monitored to increase and decrease pressure over a given treatment period. A treatment protocol program monitors and tracks the quality and quantity of brace “wear time” to provide better data for modifying and/or increasing bladder pressures and cycle times over a long-term bracing period.

More specifically, the overall system comprises a novel scoliosis brace 10 with an integrated inflatable bladder system 12, and external control device (cell phone/tablet) 14 running a proprietary control and monitoring application. Some embodiments of the system may further include a remote back-end server 16 which receives and stores patient data, operating values and long term recorded wear data for remote access by supervising physicians 18.

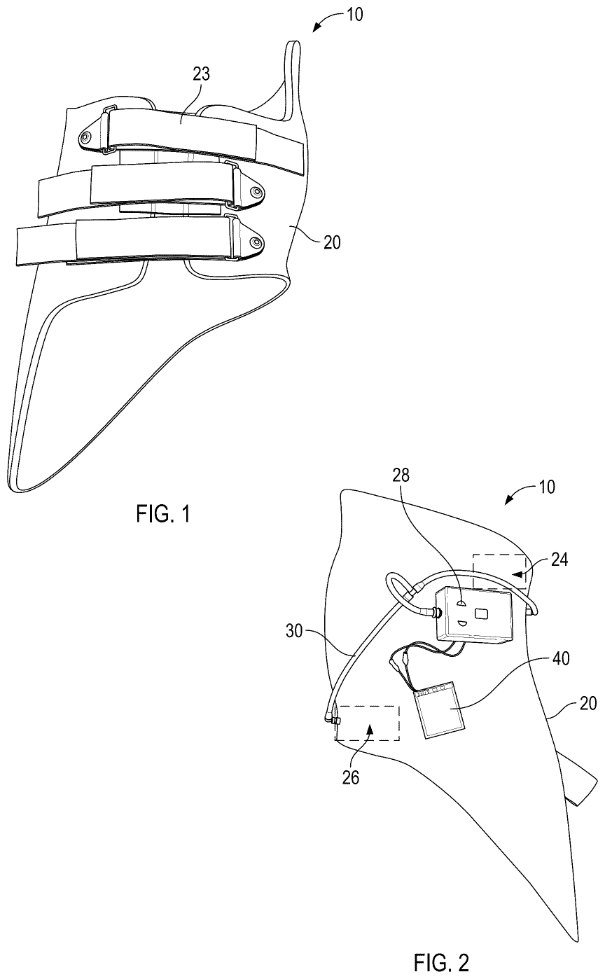

An exemplary embodiment of a scoliosis brace 10 in accordance with the teachings of the invention is illustrated in FIGS. 1-3. The scoliosis brace 10 may comprise a rigid outer shell 20, an interior padding layer 22 disposed on an inner surface of the outer shell 20 and adjustable closure straps 23. The outer shell 20 may be custom form fit and molded to the individual user to provide proper external bracing support for the interior padding and bladder components.

One or more inflatable air bladders 24, 26 of predetermined shape and size are located between the outer shell 20 and the inner foam padding 22. Placement of the bladder(s) 24, 26 between the outer shell 20 and the inner foam padding 22 distributes bladder pressure more evenly over a broader surface area and prevents uncomfortable localized pressure points which are a common cause of non-compliant wear. The shape, size and position of the inflatable bladder(s) 24, 26 within the shell 20 are configured so as to be effective for applying more evenly distributed pressure to a targeted location(s) on the spine of a patient.

In some embodiments, the bladder(s) 24, 26 may be placed into a pocket formed between two foam layers.

As noted above, the air bladders 24, 26 may be shaped corresponding to the relative location of the respective bladder within the brace. For example, an upper thoracic bladder 24 may be generally shaped as a parallelogram while a lumbar bladder 26 may be generally shaped as a triangle or tear drop with the apex pointed towards the small of the back. Various progressively sized bladders may be provided so as to be interchangeable within the system to provide adaptability to various body size and to grow with adolescent patients.

The bladder system 12 further includes an air pump 28 in fluid communication with the inflatable bladder(s) 24, 26. Movement of air from the pump 28 to the bladders 24, 26 is accomplished with supply tubes 30 which are appropriately routed to the bladder(s) 24, 26. A single pump 28 may be provided to inflate two distinct bladders 24, 26.

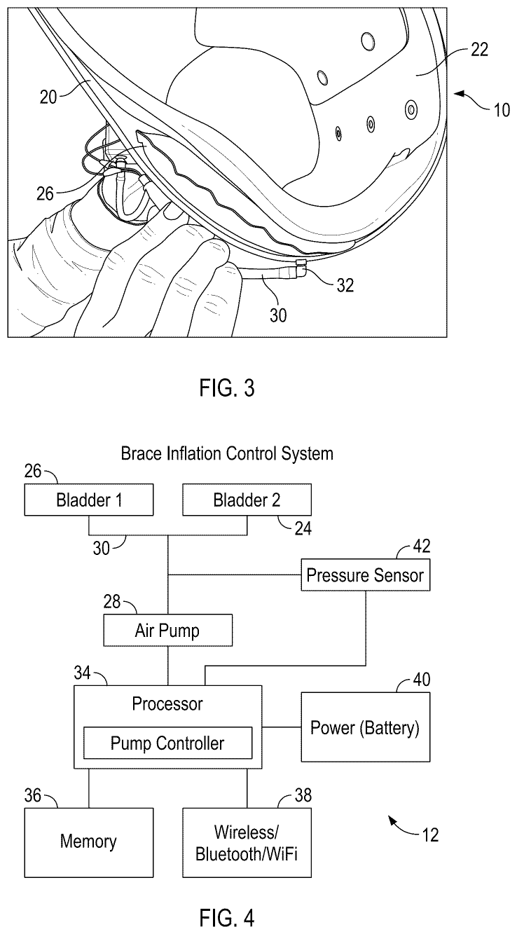

To provide interchangeability of the bladders 24, 26, the supply tube termination ends and the bladder inlet ports 32 may be provided with releasable interfitting connections (see FIGS. 2 and 3).

An exemplary device level block diagram of the bracing system electronic and control components is illustrated in FIG. 4. A microcontroller/processor 34 in communication with the air pump 28 is operable for selectively controlling the air pump 28 to inflate and deflate the bladder(s) 24, 26 according to various operating values and protocols to be described hereinafter. The processor 34 may include associated memory 36 and a wireless communication transceiver 38, such as Bluetooth or WiFi. The inflation system 12 may further include a rechargeable power source (battery) 40. A pressure sensor 42 receives input from the air supply tubes 30 and communicates real time pressure data to the processor 34 and/or to the wireless control device 14 (and processor 44) for monitoring, analytics and long term data acquisition and aggregation.

The system may further include an external control device 14, such as a mobile phone or tablet, which is in wireless paired communication with the inflatable bladder system 12. Wireless communication may be accomplished by any known wireless transceiver 50 but may preferably comprise a Bluetooth paired connection or a direct WiFi connection with the bladder inflation system 12.

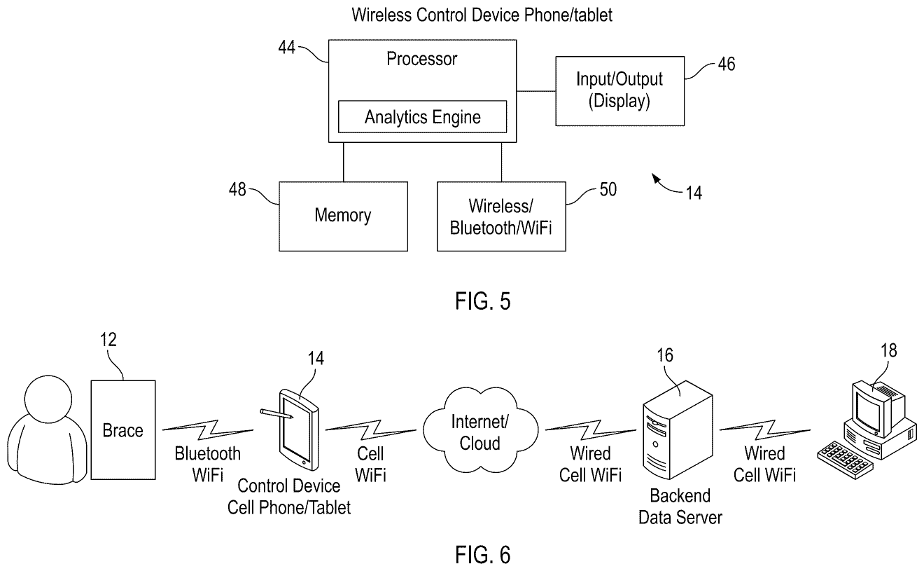

A block diagram of an exemplary control device 14 is illustrated in FIG. 5. An exemplary external control device (cell phone/tablet) 12 may comprise an input/output user interface 46 (LCD touch screen display) for receiving input from the user and displaying output, a memory 48 which may include a table of stored operating values and a processor 44 coupled to the memory 48 and the user interface 46, and which is programmed with executable instructions including an analytics engine. Pump control and pressure maintenance logic may in some embodiments be implemented in the device pump control 34 and in some embodiments may be implemented in the wireless control device 14.

Referring to FIG. 6, the system 12 may also be controlled remotely by accessing the remote server 16 directly and directing input to a specified device registered within the remote database/server system. The remote server 16 is also utilized for maintaining patient/user accounts, remotely storing aggregated user wear data for review and analysis by physicians overseeing treatment of the patient, direct remote control based on control algorithms implemented at the remote server level and for software updates.

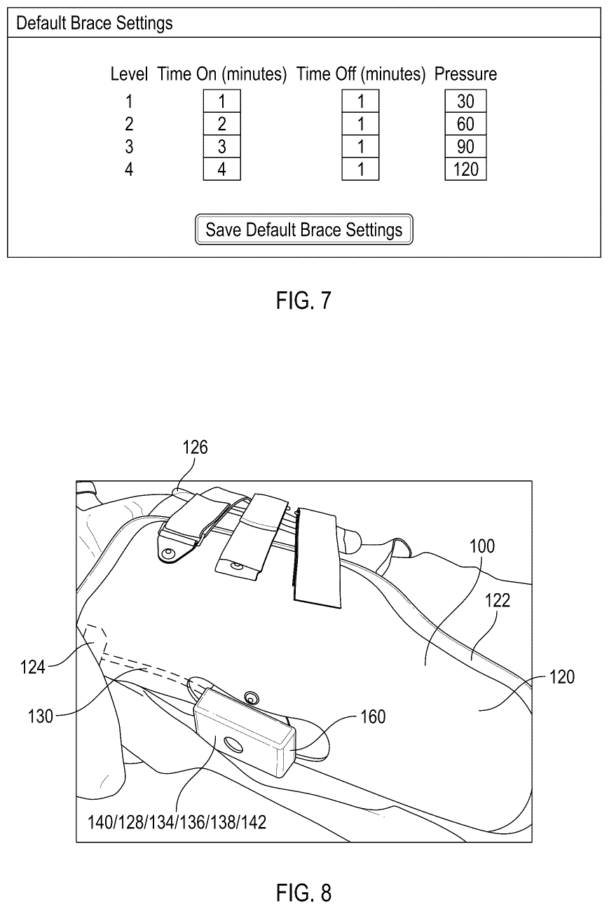

The stored operating values within the control memory 48 may include on and off cycles, on and off cycle times and progressive cycle pressures. An exemplary table of cycle times and pressure is shown in FIG. 7. The exemplary table is not intended to be limiting and may be fully customizable to individual patient needs and treatment protocols.

With respect to the operating values as noted, a critical premise of the present invention is a realization that timed cyclic pressure is more tolerable to the wearer and thus will promote more consistent and longer-term quality use, leading to improved outcomes. The operating times and pressure values can be completely customized based on individual patient needs, body type, BMI, severity of curvature, age, etc.

Generally, the methodology of the analytics system operating on the brace bladder and control device is as follows:
a) receiving input from a user to identify an operating value and to start and stop a timed wear cycle;
b) selectively controlling the air pump to cyclically inflate and deflate the bladder according to the operating value/predetermined pressure during the timed wear cycle;
c) operating a feedback loop which continuously monitors pressure of the bladder,
d) selectively controlling the air pump to maintain the predetermined pressure at a constant level based on the feedback loop;
e) continuously recording pressure values during the timed wear cycle;
f) determining an effective wear time based on changes in the recorded pressure values;
g) calculating a bracing score based on said effective wear time; and
h) displaying the bracing score at the conclusion of the timed wear cycle.

Prior art systems have attempted various methods for recording “effective wear time” but as noted above, these methods and systems relied heavily on physical switches (force sensors) and/or an honor system. The present analytics system uses the recorded pressure values from the feedback loop as a means of determining whether the brace is actually being worn. Changes in pressure on the bladder(s) 24, 26 during normal operation and normal active sleep patterns are highly indicative of actual wear time.

The bracing score is based on a 100-point system and is calculated based on an 8-hour sleep/wear cycle with the patient receiving 12.5 points per hour of normal effective wear time. A full 8-hour wear cycle would generate a score of 100, while a 7-hour wear time would generate a score of 87.5, etc. The point system gives young adults a familiar scoring system which they can easily relate to for effectiveness.

In a treatment regimen, it would be foreseen that with the bracing scores and improved wear tracking, the patient could increase the operating “level” to the next step approximately every three months with actual patient visits, or more quickly based on higher compliance score, or more slowly based on lower compliance scores. The system thus provides the physician a concrete data set of quality and quantity wear time on which to base future treatment.

As noted above, the scoliosis bracing system 12 may further comprise a remote server 16 in wireless communication with the external control device 14. Such a remote server 16 may comprise, for example, a PC or laptop with a user interface for receiving input and displaying output, a memory including a database of patient data wherein the patent data includes prescribed operating values, recorded wear data and bracing scores, a processor coupled to the memory and the user interface, and programmed with executable instructions including an analytics engine which is operable for receiving input from a user to set operating values and to retrieve recorded wear data, and for displaying output from the database based on the input.

The backend server 16 may in turn be accessed remotely from another PC or computer device 18 to provide an effective cloud-based operating system.

Referring to FIG. 8 there is shown another exemplary embodiment of a scoliosis brace 100 including bladders 124, 126 (shown in dotted line) wherein the battery 140, pump 128 and various electronics (pressure sensor 134, 142, 136, 138) are packaged within a common housing 160 mounted on the exterior of the shell 120 and the air supply tubes 130 are routed from the pump 128 into the brace shell 120 within the space between the shell 120 and the foam padding 122.

An exemplary scoliosis treatment method according to the invention may comprise the follow steps:
providing a scoliosis brace comprising,
   an outer shell; and
   a padding layer disposed on an inner surface of the outer shell,
providing an inflatable bladder system comprising:
   at least one inflatable bladder of predetermined shape and size disposed in a predetermined position between the padding layer and the outer shell,
   the shape, size and position of the inflatable bladder being effective for applying pressure to a targeted location on the spine of a patient;
   an air pump in fluid communication with the inflatable bladder; and
   a controller in communication with the air pump operable for selectively controlling the air pump to inflate and deflate the bladder;
providing an external control device in wireless communication with the inflatable bladder system, the external control device comprising;
   a user interface for receiving input and displaying output;
   a memory including a table of operating values, wherein the operating values include on and off cycles, on and off cycle times and cycle pressures; and
   a processor coupled to the memory and the user interface, and programmed with executable instructions;
starting a timed wear cycle and an operating value;
selectively controlling the air pump to cyclically inflate and deflate the bladder according to the operating value during the timed wear cycle;
monitoring a feedback loop which continuously monitors pressure of the bladder,
selectively controlling the air pump to maintain said predetermined pressure at a constant level based on said feedback loop;
continuously recording pressure values during the timed wear cycle;
stopping the timed wear cycle;
determining an effective wear time based on changes in said recorded pressure values;
calculating a bracing score based on said effective wear time; and
displaying the bracing score at the conclusion of a timed wear cycle.

An exemplary treatment method may further comprise steps including increasing the operating values at predetermined calendar/timed intervals and at least partially based on consistent repeated bracing scores over said calendar intervals.

Treatment incentives or motivations may include but are not limited to: 1) instant feedback via a daily brace score; 2) comparison to others in the study to stimulate competition between others with a similar problem (all patients are blinded to others in the study); and 3) longitudinal tracking to measure progress over time to encourage compliance over the course of brace wear.

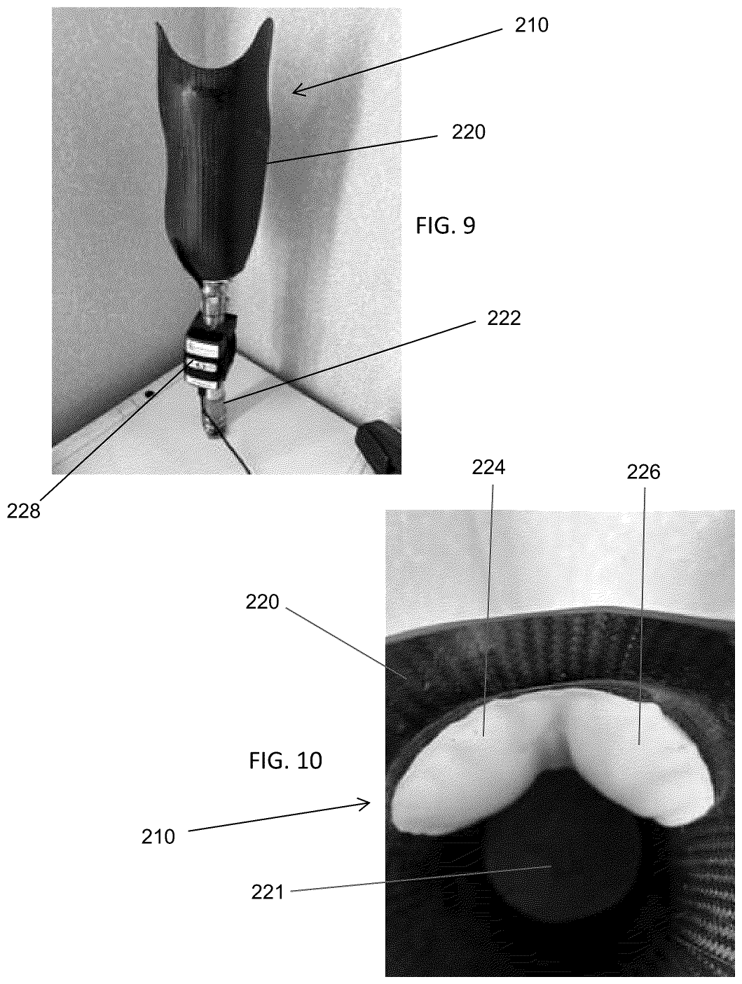

Turing to FIGS. 9-16, according to some embodiments of the invention, a novel prosthesis 210 includes inflatable pressure bladders which are user controlled and monitored to automatically inflate and deflate to maintain a user-set pressure as the volume of the limb increases and decreases. A control program also monitors and tracks pressure data for review by prosthesis clinicians.

More specifically, the overall system comprises a novel prosthesis 210 with an integrated inflatable bladder system 212, and external control device (cell phone/tablet) 214 running a proprietary control and monitoring application. Some embodiments of the system may further include a remote back-end server 216 which receives and stores patient data, operating values and long term recorded pressure data for remote access by supervising physicians 218.

An exemplary embodiment of a prosthesis 210 in accordance with the teachings of the invention is illustrated in FIGS. 9-10. The prosthesis 210 may comprise a rigid outer shell 220 having an interior socket 221 which may be custom form fit and molded to the individual user to provide proper external support for the bladder components. A support pylon 222 extends from the shell. The illustrated prosthesis 210 comprises a below-knee (BK) prosthesis. However, the invention should not be considered to be limited any particular socket configuration.

One or more inflatable air bladders 224, 226 of predetermined shape and size are located within the outer shell 220. Placement of the bladder(s) 224, 226 distributes bladder pressure more evenly over a broader surface area and prevents uncomfortable localized pressure points which are a common cause of discomfort. The shape, size and position of the inflatable bladder(s) 224, 226 within the shell 220 are configured so as to be effective for applying more evenly distributed pressure to a targeted location(s) on the residual limb of a patient. Because the bladders 224, 226 are custom made for each individual patient, they more securely surround the anatomy with the bladder and when it is inflated it “hugs” the tibia (BK-below knee) or the femur (AK-Above knee) which is an effective control of the anatomy that can't be achieved with socket modifications alone. The inflated bladder thus softly controls the anatomy and prevents bruising or trauma to the limb because of the constant motion inside the socket.

Some embodiments may include interior padding or foam layers. In some embodiments, the bladder(s) 224, 226 may be placed into a pocket formed between two foam layers.

As noted above, the air bladders 224, 226 may be shaped corresponding to the relative location of the respective bladder within the prosthesis socket. Various sized bladders may be provided so as to be interchangeable within the system to provide adaptability to various intended activities of the patient.

The bladder system 212 further includes a battery-powered air pump 228 in fluid communication with the inflatable bladder(s) 224, 226. The pump 228 may be located in a housing configured in line with the support pylon 222 below the socket. Movement of air from the pump 228 to the bladders 224, 226 is accomplished with supply tubes 230 (not illustrated) which are appropriately routed to the bladder(s) 224, 226 within the socket through the interior of the pylon 222. A single pump 228 may be provided to inflate two distinct bladders 224, 226.

To provide interchangeability of the bladders 224, 226, the supply tube termination ends and the bladder inlet ports may be provided with releasable interfitting connections (not shown).

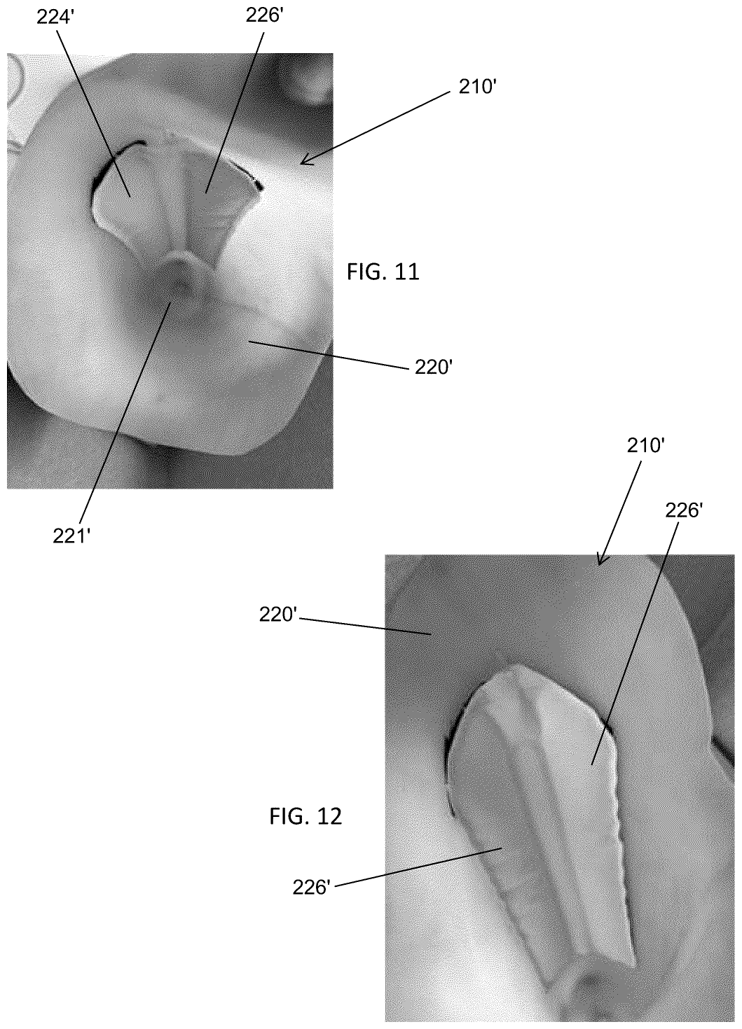

Turning briefly to FIGS. 11 and 12, an above-knee (AK) prosthesis 210′ is illustrated, and comprises similar socket 220′ and bladder components 224′, 226′.

Referring now to FIG. 14, an exemplary device level block diagram of the inflation system electronic and control components is illustrated. A microcontroller/processor 234 in communication with the air pump 228 is operable for selectively controlling the air pump 228 to inflate and deflate the bladder(s) 224, 226 according to various operating values and protocols to be described hereinafter. The processor 234 may include associated memory 236 and a wireless communication transceiver 238, such as Bluetooth or WiFi. The inflation system 212 may further include a rechargeable power source (battery) 240.

A pressure sensor 242 receives input from the air supply tubes 230 and communicates real time pressure data to the processor 234 and/or to the wireless control device 214 (and processor 244) for monitoring, analytics and long term data acquisition and aggregation.

The system may further include an external control device 214 (See FIG. 5), such as a mobile phone or tablet, which is in wireless paired communication with the inflatable bladder system 212. Wireless communication may be accomplished by any known wireless transceiver 250 but may preferably comprise a Bluetooth paired connection or a direct WiFi connection with the bladder inflation system 212.

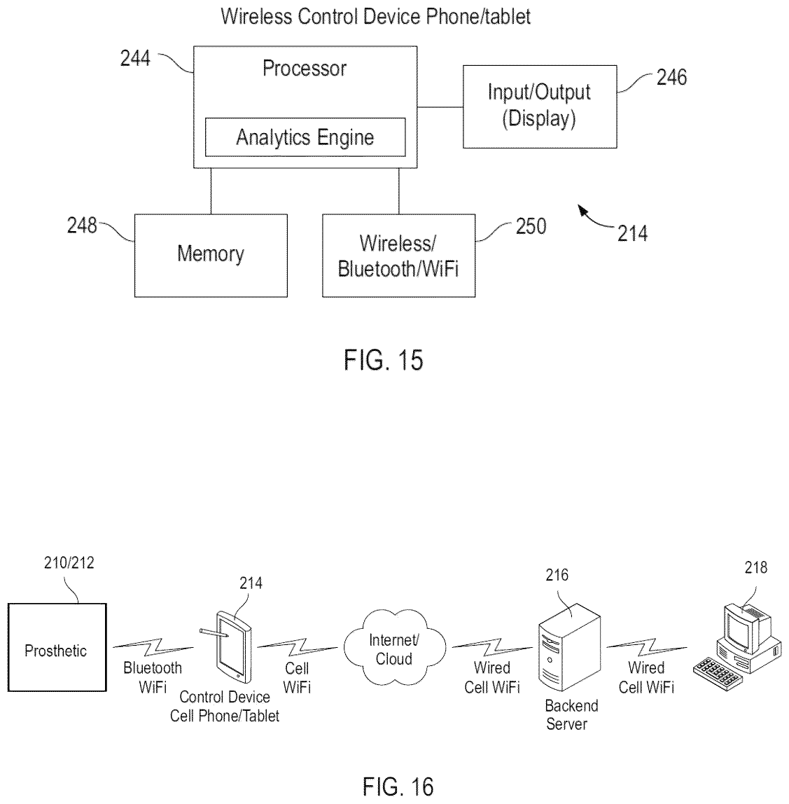

A block diagram of an exemplary control device 214 is illustrated in FIG. 15. An exemplary external control device (cell phone/tablet) 212 may comprise an input/output user interface 246 (LCD touch screen display) for receiving input from the user and displaying output, a memory 248 which may include a table of stored operating values and a processor 244 coupled to the memory 248 and the user interface 246, and which is programmed with a control application with executable instructions. Pump control and pressure maintenance logic may in some embodiments be implemented in the device pump control 234 and in some embodiments may be implemented in the wireless control device 214.

Referring to FIG. 16, the system 212 may also be controlled remotely by accessing the remote server 216 directly and directing input to a specified device registered within the remote database/server system. The remote server 216 may also be utilized for maintaining patient/user accounts, remotely storing aggregated user wear data for review and analysis by physicians overseeing treatment of the patient, direct remote control based on user input and/or control algorithms implemented at the remote server level, and for software updates.

As noted above, the system 212 may further comprise a remote server 216 in wireless communication with the external control device 14. Such a remote server 216 may comprise, for example, a PC or laptop with a user interface for receiving input and displaying output, a memory including a database of patient data wherein the patient data includes recorded wear and pressure data, a processor coupled to the memory and the user interface.

The server 216 may in turn be accessed remotely from another PC or computer device 218 to provide an effective cloud-based operating and monitoring system.

As the patient wears the prosthetic during the course of the day, the direct pressure is transmitted back to the phone and stored there. Once the patient is in proximity to a local network that stored information is uploaded to a secure cloud based server. Retrieval of that information by the prosthetist or researchers is obtained through access to the secure cloud based server via the web. Remote patient monitoring is an automatic process. No response or intervention by the patient is necessary except to connect their cell phone to their local network. Once that is done data collection is automatically uploaded to the cloud based server.

With respect to the patient operation of the system, the operating pressure values can be completely customized based on individual patient needs and comfort.

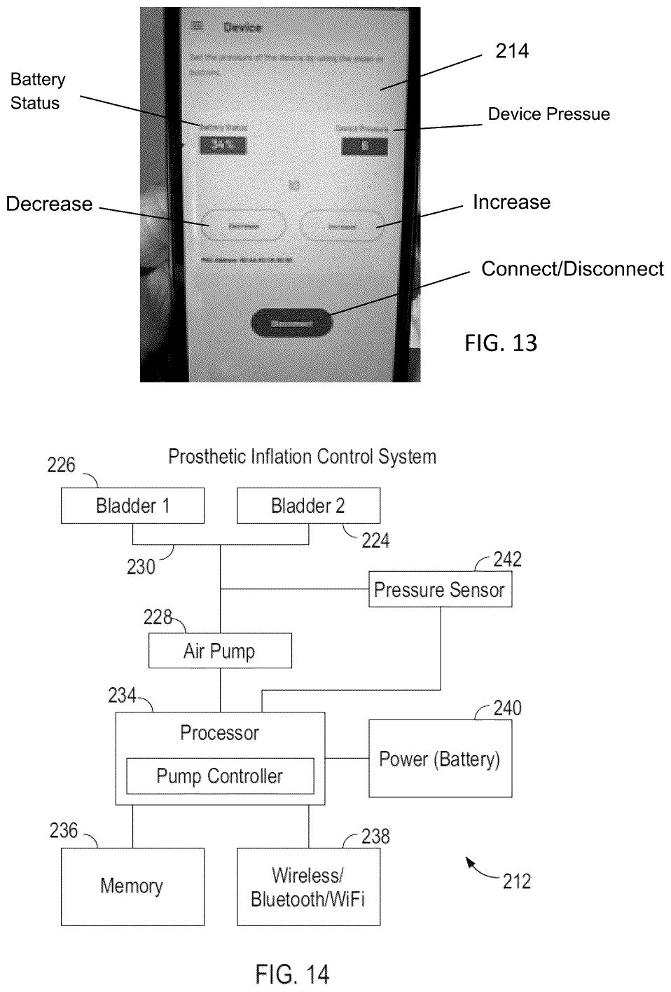

The application on the smartphone (See FIG. 13) sends a signal to the inflation system which in turn inflates the air bladders to the desired pressure set by the patient based on their own individual needs. The inflation system maintains the set pressure in the bladder within a few millimeters of mercury (mm HG). As a result, if the patient's limb shrinks during the day, the bladder will inflate to maintain the pressure set, and vice versa. This equates to automatic “volume” control within the prosthetic socket. Once the patient sets the desired pressure, the system maintains that same pressure throughout the course of the day, until changed or disconnected from the remote application system.

If the patient is sitting, the pressure can be selectively decreased by the patient to allow the socket to be more comfortable. The control of the pressure is directly in the patient's hands.

The custom bladders are thus highly effective for anatomically stabilizing the anatomy of the residual limb. As muscles atrophy because of inactivity the muscle mass around becomes less firm and the tibia and femur is allowed to float with in the socket. The bladder is constructed to “trap” or enclose the tibia or femur in a soft way and limit this movement with in the prosthetic socket. The bladder system will give the patient more control over their prosthesis and make gait more efficient. Furthermore, patients will use their prosthesis more. All of this can be validated by data collection.

The combination of the two features means that patient can adjust the volume of the socket according to their personal preference and the activity they plan to do. For highly active activities: hiking, jogging, running other sports the patient can increase the pressure. For sitting is a car for a long period of time, the patient can decrease or disconnect the device. This means that there will be less trips to the prosthetist to have the socket adjusted, and that the patient will have a better fitting, more efficient socket.

While there is shown and described herein certain specific structures embodying various embodiments of the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. A prosthetic socket system comprising:
a prosthetic socket comprising,
an outer shell;
an inflatable bladder system comprising:
at least one inflatable bladder of predetermined shape and size disposed within the outer shell,
an air pump in fluid communication with the at least one inflatable bladder; and
a pump controller in communication with the air pump operable for selectively controlling the air pump to inflate and deflate the at least one bladder; and
an external control device in wireless communication with the inflatable bladder system, said external control device comprising;
a user interface for receiving input and displaying output;
a memory including a table of operating values;
a processor coupled to the memory and the user interface, and programmed with executable instructions including an analytics engine which is operable for
a) receiving input from a user to set an operating pressure value;
b) selectively controlling the air pump to cyclically inflate and deflate the bladder according to said operating value;
c) operating a feedback loop which continuously monitors pressure of the bladder,
d) selectively controlling the air pump to maintain said predetermined pressure at a constant level based on said feedback loop; and
e) continuously recording pressure values during said timed wear cycle.

2. The prosthetic socket system of claim 1 wherein the prosthetic socket further comprises a padding layer disposed on an inner surface of the outer shell.

3. The prosthetic socket system of claim 1 further comprising a remote server in wireless communication with said external control device, said remote server comprising:
a user interface for receiving input and displaying output;
a memory including a database of patient data wherein said patent data includes user set operating values and recorded wear data;
a processor coupled to the memory and the user interface, and programmed with executable instructions including an analytics engine which is operable for
a) receiving input from a user to set operating values and retrieve recorded wear data; and
b) displaying output from said database based on said input.

4. The prosthetic socket system of claim 2 wherein the at least one inflatable bladder is disposed in a predetermined position between the padding layer and the outer shell,
said shape, size and position of said inflatable bladder being effective for applying pressure to a targeted location on limb of a patient.

5. The prosthetic socket system of claim 1 wherein the inflatable bladder is generally parallelogram in shape.

6. The prosthetic socket system of claim 4 wherein the inflatable bladder is generally parallelogram in shape.

7. The prosthetic socket system of claim 1 wherein the prosthetic socket further comprises a support pylon and wherein the air pump and pump controller are located within the support pylon.
